(19) United States
(12) Patent Application Publication
(10) Pub. No.: US 2026/0199340 A1
(43) Pub. Date: Jul. 16, 2026

(54) STABLE AQUEOUS INJECTABLE FORMULATION OF FLUCYTOSINE

(71) Applicant: CIPLA LIMITED, Mumbai, Maharashtra (IN)

(72) Inventors: Pradeep BHADAURIA, Weston, FL (US); Arunprasath KALIAPERUMAL, Weston, FL (US); Tushar RAJYAGURU, Bhavnagar, Gujarat (IN); Harshad HARDE, Nashik, Maharashtra (IN); Sandeep KHAIRNAAR, Nashik, Maharashtra (IN)

(21) Appl. No.: 19/138,052

(22) PCT Filed: Dec. 22, 2023
(86) PCT No.: PCT/IN2023/051212

§ 371 Date: Jun. 11, 2025

(30) Foreign Application Priority Data
Dec. 26, 2022 (IN) 202221075574

(51) Int. Cl. A61K 31/513; A61K 9/00; A61K 9/08; A61K 47/02

(52) U.S. Cl. CPC A61K 31/513; A61K 9/0019; A61K 9/08; A61K 47/02

(57) ABSTRACT

The present invention relates to sterile ready to use aqueous pharmaceutical formulation comprising flucytosine and pharmaceutically acceptable excipients wherein the pH of formulation is maintained between 6.0 to 8.0. The invention further relates to the method of manufacture and sterilization of such formulations and using the formulation for treating systemic yeast and fungal infections.

### FIELD OF INVENTION

The present invention relates to sterile ready to use aqueous pharmaceutical formulation comprising flucytosine and pharmaceutically acceptable excipients wherein the pH of formulation is maintained between 6.0 to 8.0. The invention further relates to the method of manufacture and sterilization of such formulations and using the formulation for treating systemic yeast and fungal infections.

### BACKGROUND OF INVENTION

Injectable solutions are intended for administration to the human body by intravenous, intramuscular, or subcutaneous injection. Advantages of injectables over oral dosage forms include fast onset of action, reproducible PK/efficacy profile, high bioavailability as a result of bypassing the oral absorption barrier, and suitability of administration under hospital setting. Thus, in some circumstances, depending on the severity of disease, patient condition, dosing schedules, patient compliance, ease of caretaker and hospital settings, injectable solutions are preferred than oral dosage forms.

However, development of parenteral dosage form is not easy and it requires selection of excipients such as buffer, pH, stabilizers, tonicity adjusting agents, antioxidants, preservatives, and solubility and/or viscosity-enhancing agents which are compatible with the drug as well as provide the desired qualities to the formulation at an optimum concentration. The main challenges of parenteral dosage forms are achievement of formulation stability, compatibility of drug substance with packaging components, and sufficient drug concentration within a reasonable pH range and without using high excipient levels that may cause blood incompatibility and tissue irritation issues. These requirements necessitate a comprehensive characterization of drug.

In order to meet the safety, efficacy, and quality standards of parenteral dosage forms, injectable solutions have to be sterile, low pyrogen, and meet the requirements of compendia specifications, such as >90% label claim, related substance level lower than the toxicity qualified level, content uniformity, pH, osmolality, particulate matter, essentially free of visual foreign matter, etc.

It is known that the pH is one of the critical aspects of parenteral formulation, which should have a target pH as much as possible close to physiological pH. An ionizable compound can be solubilized to the desired concentration by pH adjustment. The acceptable range is pH 2-11 for intravenous and intramuscular injection and pH 4-9 for subcutaneous injection due to potential irritation issue. The solution pH can be controlled by a buffer, such as acetate, phosphate, citrate, histidine, TRIS, and others, to an ideal range of pH 5-8. The buffer capacity should be kept to a minimum to enable body fluid to adjust the formulation pH quickly to physiological pH; otherwise, an irritation at the injection site may be present. For IV infusion, the solution pH is typically controlled by the salt form of the drug or by acids and bases without buffer due to in vivo tolerability considerations. In some cases, a compromise may be required between solubility and stability of the drug substance in order to find a target formulation pH in which both drug loading and product stability are acceptable.

One drug which is used in the management and treatment of systemic and severe candida and cryptococcus infections is Flucytosine (5FC) which is available in the form of oral capsules (ANCOBON®) in US as well as in the form of solution for infusion (ANCOTIL) for intravenous or intraperitoneal administration in some countries like Europe and Australia. The dosing schedule of capsules is usually 50 to 150 mg/kg of body weight per day, taken divided doses at 6-hour intervals which may lead to a higher workload for clinical personnel, poor patient compliance, thereby increasing the probability of administering suboptimal doses, missing the doses and ultimately contributing to the emergence of resistant fungal strains. Thus, the solution for infusion of flucytosine may be preferred for some critically ill patients in special circumstances. ANCOTIL is available in packs of 5 glass bottles of 250 ml. Each infusion bottle of flucytosine contains 2.5 g in 250 ml (1 g in 100 ml) of flucytosine and other ingredients such as Sodium chloride, tromethamine, hydrochloric acid, and water for injections, maintaining the optimum pH of 6-8. There is a strict storage temperature requirements for ANCOTIL to avoid precipitation as well as decomposition during long term storage. It is known that “Ancotil for Infusion should be stored between 18° C. and 25° C. If stored below 18° C., precipitation of flucytosine substance may occur.

Ancotil injection contains tromethamine as buffer. In some injectable formulations, hydrolysis takes place in presence of tromethamine thus leading to generation of undesired impurities and degradation of product. For patients who are allergic to tromethamine, local irritation and tissue inflammation or infection at the site of injection, a febrile response, chemical phlebitis, venospasm, hypervolemia, and iv thrombosis maybe caused by administration of solution comprising tromethamine. The inventors of present invention have provided a sterile ready to use aqueous pharmaceutical formulation of flucytosine with minimum excipients while maintaining the desired qualities of the formulation. Through such rigorous experimentation which involved the compatibility studies with various excipients including buffers, pH adjusters, tonicity adjusting agents at various concentrations, inventors have found that the formulation remains stable even without addition of buffer. Thus, the inventors of present invention have provided sterile ready to use aqueous pharmaceutical formulation comprising flucytosine or its pharmaceutically acceptable salt and pharmaceutically acceptable excipients, optionally buffer, maintaining pH of 6-8. Also, in an embodiment, the inventors of present invention have provided sterile ready to use aqueous pharmaceutical formulation comprising flucytosine or its pharmaceutically acceptable salt and pharmaceutically acceptable excipients, free of buffer and yet maintaining the pH of 6-8 and stability of formulation throughout the shelf life.

Further, the cytotoxic and teratogenic degradation product 5-fluorouracil (5FU) may be present in considerable amounts in the product, due to both impurities in the raw material and the formation of 5FU upon sterilisation and inappropriate storage. Since the proposed formulation is an injection product, it is desired that it should be free from visible particulate matters, any precipitates, and impurities. Thus, the manufacturing process of formulation, sterilisation process of formulation and storage of formulation also plays a critical role. Exemplary sterilisation procedures include terminal moist heat sterilization, ethylene oxide, radiation (i.e., gamma and electron beam), and aseptic processing techniques. During the development of formulation of present invention, the inventors subjected the flucytosine solution for infusion to the typically recommended method of sterilisation by regulatory authorities i.e., terminal sterilisation method for sealed packages of large volume parenterals (LVPs). However, the inventors found that the flucytosine solution for infusion was unable to withstand the terminal sterilization condition by autoclaving at higher temperatures and resulted in rise of cytotoxic impurity 5FU above acceptable limits.

Therefore, considering the impurity generation of 5-fluorouracil (5FU) and strict storage conditions, development of a sterile, stable, ready-to-use formulation of flucytosine, which is easy to administer to the patient, is challenging. The inventors of present invention have provided a sterile formulation which will overcome the above described shortcomings of capsules and vials of flucytosine, do not have undesirable amount of impurity as well as can be transported and stored easily and which can be administered to a patient by intravenous or intraperitoneal without any intermediate step(s) of dilution/reconstitution or any other type of manipulation(s), which could otherwise compromise the sterility of the solution in the infusion container or could result in an error in dosing to a patient in need thereof.

### Object of the Invention

An object of the present invention is to provide a sterile ready to use aqueous pharmaceutical formulation comprising flucytosine or its pharmaceutically acceptable salts in a therapeutically effective amount and pharmaceutically acceptable excipients having a pH range of from about 6.0 to about 8.0.

Another object of present invention is to provide a sterile ready to use aqueous pharmaceutical formulation comprising flucytosine or its pharmaceutically acceptable salts in a therapeutically effective amount and pharmaceutically acceptable excipients having a pH range of from about 6.0 to about 8.0, wherein said aqueous solution is free of any buffer.

Particularly, the object of present invention is to provide a sterile ready to use aqueous pharmaceutical formulation comprising (a) flucytosine (b) tonicity adjusting agent, (c) pH adjusting agent and (d) aqueous vehicle, optionally buffering agent, having a pH range of from about 6.0 to about 8.0 wherein the formulation being aseptic filled and stored in storage container suitable for parenteral administration.

An object is also to provide a method of manufacture and sterilisation process of formulation of present invention comprising flucytosine or its pharmaceutically acceptable salts, and pharmaceutically acceptable excipients.

Another object of the present invention is to provide formulation of flucytosine which is stable for long shelf life.

### SUMMARY OF THE INVENTION

The present invention relates to sterile ready to use aqueous pharmaceutical formulation comprising flucytosine and pharmaceutically acceptable excipients wherein the pH of formulation is maintained between 6.0 to 8.0. The invention further relates to the method of manufacture and sterilization of such formulations and using the formulation for treating systemic yeast and fungal infections.

The present invention relates to a sterile ready to use aqueous pharmaceutical formulation comprising flucytosine or its pharmaceutically acceptable salts thereof, wherein the pharmaceutical formulation is free of buffering agent.

In one embodiment, the formulation of the present invention further comprises a pharmaceutically acceptable excipient selected from a tonicity adjusting agent, and a pH adjusting agent.

In another embodiment, the formulation of the present invention comprises about 5 mg/ml to about 20 mg/ml flucytosine or its pharmaceutically acceptable salts thereof.

In a preferred embodiment, the formulation of the present invention comprises about 10 mg/ml flucytosine or its pharmaceutically acceptable salts thereof.

In one other embodiment, the formulation of the present invention comprises sodium chloride as tonicity adjusting agent.

In another embodiment, the formulation of the present invention further comprises a pH adjusting agent. In an embodiment, the formulation of the present invention has a pH between about 6.0 to about 8.0. In a preferred embodiment, the formulation of the present invention has a pH of about 7.5.

In a further embodiment, the formulation of the present invention is manufactured by aseptic fill finish method.

In an embodiment, the formulation of the present invention, when stored for 24 months at 25° C./60% RH contains no more than 0.7% of 5-fluorouracil impurity.

In one more embodiment, the formulation of the present invention, when stored for 24 months at 25° C./60% RH contains no more than 0.2% of ethenoflucytosine impurity.

Another embodiment relates to use of the formulation of the present invention in treatment of systemic yeast and fungal infections.

A further embodiment relates to a sterile ready to use aqueous pharmaceutical formulation comprising about 5 mg/ml to about 20 mg/ml flucytosine or its pharmaceutically acceptable salts thereof, about 8.05 mg/ml sodium chloride, and optionally a pH adjusting agent, wherein the pharmaceutical formulation is free of buffering agent. In a preferred embodiment, the formulation of the present invention comprises about 10 mg/ml flucytosine or its pharmaceutically acceptable salts thereof.

Another embodiment relates to a sterile ready to use aqueous pharmaceutical formulation comprising about 5 mg/ml to about 20 mg/ml flucytosine or its pharmaceutically acceptable salts thereof, about 6.5 mg/ml sodium chloride, and optionally a pH adjusting agent, wherein the pharmaceutical formulation is free of buffering agent. In a preferred embodiment, the formulation of the present invention comprises about 10 mg/ml flucytosine or its pharmaceutically acceptable salts thereof.

According to an aspect of present invention, there is provided a sterile ready to use aqueous pharmaceutical formulation comprising flucytosine or its pharmaceutically acceptable salts in a therapeutically effective amount and pharmaceutically acceptable excipients having a pH range of from about 6.0 to about 8.0.

Another aspect of present invention is to provide a sterile ready to use aqueous pharmaceutical formulation comprising flucytosine or its pharmaceutically acceptable salts in a therapeutically effective amount and pharmaceutically acceptable excipients having a pH range of from about 6.0 to about 8.0 wherein said aqueous solution is free of any buffer.

Particularly, the aspect of present invention is to provide a sterile ready to use aqueous pharmaceutical formulation comprising (a) flucytosine (b) tonicity adjusting agent, (c) pH adjusting agent and (d) aqueous vehicle, optionally buffering agent, having a pH range of from about 6.0 to about 8.0 wherein the formulation is aseptically filled and stored in storage container suitable for parenteral formulations.

An aspect is also to provide a method of manufacture and sterilisation process of formulation of present invention comprising flucytosine or its pharmaceutically acceptable salts, and pharmaceutically acceptable excipients.

Another aspect of the present invention is to provide formulation of flucytosine which is stable for long shelf life.

### DETAILED DESCRIPTION OF THE INVENTION

The present invention relates to sterile ready to use aqueous pharmaceutical formulation comprising flucytosine and pharmaceutically acceptable excipients wherein the pH of formulation is maintained between 6.0 to 8.0. The invention further relates to the method of manufacture and sterilization of such formulations and using the formulation for treating systemic yeast and fungal infections.

Flucytosine (5FC) is an antifungal medication used in the management and treatment of systemic and severe candida and cryptococcus infections. Flucytosine is 5-fluorocytosine chemically, a fluorinated pyrimidine which is related to fluorouracil and floxuridine. Its structural formula is:

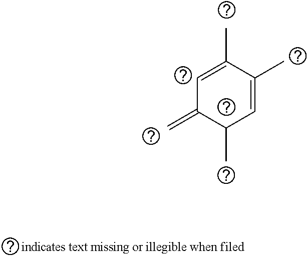

Flucytosine (5FC) is a prodrug of 5-Flurouracil (5FCU). 5FC is taken up by fungal cells by cytosine permease and converted into fluorouracil (5FU) by fungal cytosine deaminase. Side effects of 5FC include nausea, vomiting, diarrhea, bone marrow depression, and hepatotoxicity. The latter two are thought to be due to effects of 5FU as it is toxic in nature at higher doses. There is always a need to control the conversion of 5FC into 5FU in vivo as well as during the shelf life of formulation.

The terms “composition” or “formulation” or “dosage form” have been employed interchangeably for the purpose of the present invention and mean that it is a pharmaceutical formulation which is suitable for administration to a patient or subject. The subject can be an animal, preferably a mammal, more preferably a human.

The term “stable formulations” refers to flucytosine formulations of present invention that are physically as well as chemically stable as demonstrated by compliance to acceptable specification when the formulation is stored at ambient temperature, such as between about 18° C. and about 25° C., for a commercially reasonable period of time, such as at least about 1 day, at least about 1 week, at least about 1 month, at least about 3 months, at least about 6 months, at least about 1 year, or at least about 2 years. Suitably, the solution of flucytosine of present invention remains physically stable, with no precipitation or crystallization or color change upon storage and the value of percentage transmittance of the solution remaining greater than 90%, preferably greater than 95% for the shelf life period of 18-24 months when stored at room temperature. Suitably, the solution of flucytosine remains chemically stable when stored at room temperature (about to 18° C. about 25° C.), wherein various parameters such as the drug content (assay of flucytosine) and content of related substances, i.e. known and unknown impurities remains within specified limits such as those specified according to ICH guidelines, upon storage for prolonged period of time such as for at least 12 months, preferably for 18 months, more preferably 24 months or longer. The formulation of the present invention is substantially free of impurities. For purposes of the present invention, “substantially free of impurities” shall be understood to include flucytosine containing formulations in which the amount of total impurities is less than about 5% of the sum of peak areas of all degradants, as calculated on a normalized peak area response (“PAR”) basis as determined by high performance liquid chromatograph (“HPLC”) after a period of about 18 months at a temperature of from about 5° C. to about 25° C. The amount of impurities is further calculated as being based upon the original amount of flucytosine (or salt thereof) being present in the composition or formulation. Preferably, the said stable formulations of flucytosine prevent degradation of flucytosine such that not more than 2%, not more than 1%, not more than 0.4%, or not more than 0.2% of flucytosine impurity or impurities are formed over the storage period. In yet another preferred embodiment the value of assay of flucytosine remains within the specified limit of 90-110% by weight of the label claim; the total impurities remain below 2.0%, preferably below 1.0% over shelf life.

In some preferred aspects of the invention, the time for which long term storage is contemplated include periods of at least about 18 months or longer with such that the formulation is substantially free of impurities when stored at room temperature.

The term “ready-to-use” refers that formulations of the present invention are premixed formulations that are suitable for administration to a patient without dilution or without any manipulation upon removing the formulations from a sealed packaging container or vessel.

The term “sterile” formulation, as used in the context of this application, means a formulation that has been brought to a state of sterility and has not been subsequently exposed to microbiological contamination, i.e., the container holding the sterile formulation has not been compromised. Sterile formulations are generally prepared by pharmaceutical manufacturers in accordance with current Good Manufacturing Practice (cGMP) regulations of the U.S. Food and Drug Administration.

The term “aseptic fill finish”, as used herein means a process of aseptic sterilization followed by filling under aseptic conditions into a pre-sterilized container. Aseptic sterilization preferably includes passing the formulation through a sterile filter for e.g., 0.22 μm to yield a sterile formulation. Filling of the sterile formulation into pre-sterilized containers under aseptic conditions completes the aseptic fill finish process.

The inventors of the present invention have rigorously conducted experiments with the sterilization process to maintain and control the levels of the impurities, especially the 5-fluorouracil (5-FU) and the ethenoflucytosine impurity within limits.

5-fluorouracil (5-FU) is a cytotoxic compound and is not desired in high amounts. The structure of 5-fluorouracil is as given below:

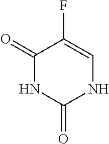

5-fluorouracil (5-FU) is a pyrimidine analogue that competitively inhibits the enzyme thymidylate synthase (TS), thereby creating a thymine deficiency and resulting in inhibition of deoxyribonucleic acid (DNA) synthesis and cytotoxicity. It also inhibits, to a lesser extent, the formation of ribonucleic acid (RNA). These effects are most marked in rapidly growing cells and may lead to cell death. It has been observed that flucytosine is closely related to 5-fluorouracil, and there is a possibility of 5-fluorouracil getting generated as an impurity over the course of time or during manufacturing of flucytosine formulation.

Owing to the cytotoxic nature of the 5-fluorouracil (5-FU) compound, it is of utmost importance to monitor levels of 5-fluorouracil (5-FU) over time, in a formulation containing flucytosine. The inventors of the present invention have rigorously evaluated different sterilization conditions and its effect on level of 5-fluorouracil (5-FU) over time.

Another impurity of particular importance is the ethenoflucytosine impurity. The chemical name of the ethenoflucytosine impurity is 8-fluoroimidazo[1,2-c] pyrimidin-5 (6H)-one and has the below structure:

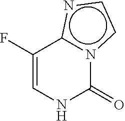

The inventors of the present invention believe to have identified this ethenoflucytosine impurity and through experimentation have been successful to maintain and control the level of this impurity over time. The inventors believe that this impurity has not been reported previously for any flucytosine formulation, let alone, any means for controlling and maintaining the levels.

The inventors of the present invention have evaluated different sterilization methods and conditions and have found that the compositions of the present invention when sterilized by aspetic fill finish method demonstrate lower levels of 5-fluorouracil (5-FU) and the ethenoflucytosine impurity. In one embodiment, the formulation comprising flucytosine or its pharmaceutically acceptable salts thereof, when stored for 24 months at 25° C./60% RH contains no more than 0.7% of 5-fluorouracil impurity. In one more embodiment, the formulation comprising flucytosine or its pharmaceutically acceptable salts thereof, when stored for 24 months at 25° C./60% RH contains no more than 0.2% of ethenoflucytosine impurity.

The present invention relates to a sterile ready to use aqueous pharmaceutical formulation of flucytosine. The pH of formulation of present invention is critical in achieving the stability of the aqueous formulation. The formulation may tend to produce undesired impurity, precipitate, produce visible particles and thus degrade at a pH less than 6.0 and more than 8.0.

The present invention provides sterile ready to use aqueous pharmaceutical formulation comprising flucytosine or its pharmaceutically acceptable salts, and pharmaceutically acceptable excipients having a pH range of from about 6.0 to about 8.0 in a container suitable for storge and administration by intravenous injection route.

In an embodiment, the formulation of present invention comprises:
(a) flucytosine or its pharmaceutically acceptable salt,
(b) tonicity adjusting agent,
(c) pH adjusting agent and
(d) aqueous vehicle,
optionally buffering agent,
   wherein the formulation has a pH from about 6.0 to about 8.0, and wherein the formulation is manufactured by aseptic fill finish and filled in a container suitable for parenteral administration.

In one embodiment, the formulation of present invention is free of any buffer. In an embodiment, the formulation of present invention comprises:
(a) flucytosine or its pharmaceutically acceptable salt,
(b) tonicity adjusting agent,
(c) pH adjusting agent and
(d) aqueous vehicle,
   wherein the formulation has a pH from about 6.0 to about 8.0, and wherein the formulation is manufactured by aseptic fill finish and filled in a container suitable for parenteral administration.

In an embodiment of the present invention, there is provided a sterile ready to use aqueous pharmaceutical formulation of flucytosine ready for direct administration to the patient in need thereof without further dilutions.

The formulation of present invention comprises flucytosine or its pharmaceutically acceptable salt in an amount of 5 mg/mL to 20 mg/mL, preferably 10 mg/mL of the total formulation.

The stable injectable pharmaceutical formulations of the present invention include one or more pharmaceutically acceptable excipients.

The parenteral administration of solutions requires that they be adjusted to isotonicity. The isotonicity of the formulation may be obtained by adding an astutely calculated quantity of tonicity agents. As per an embodiment, suitable tonicity adjusting agent which may be used in the formulation of present invention include, but not limited to, sodium chloride, potassium chloride, calcium chloride, sodium hydroxide, potassium hydroxide, dextrose, sodium carbonate, meglumine, sodium lactate, Ringer's solution and lactated Ringer's solution. The preferred tonicity adjusting agent present in formulation of present invention is sodium chloride. The tonicity agent is present in the amount from about 1.0 mg/ml to about 20 mg/ml, preferably from about 5.0 mg/ml to 10 mg/ml, more preferably about 6.5 mg/ml or about 8.05 mg/ml.

According to a particular embodiment, the formulations may optionally comprise at least one pH adjusting agent such as sodium hydroxide or hydrochloric acid to adjust the pH to around 6.0 to 8.0. In the case where the buffering agent is constituted of acetic acid/sodium acetate, using additional acetic acid as a pH adjuster is advantageous.

The pH of the formulation changes according to the amount of buffer used. It is preferable to achieve a pH level of between 5.0 and 8.0, preferable to have a pH of about 6.0 to about 8, and most preferable to develop a formulation with a pH of about 7.5.

According to the present invention, the formulation of present invention may optionally contain a buffering agent to maintain the pH. The buffering agent may comprise at least one buffering agent selected from the group consisting of citrate (sodium or potassium), acetate, phosphate, malate, lactate, glutamate, titrate, benzoate, glycine, tromethamine (Tris) and mixtures thereof. The most preferably used buffering agent is tromethamine.

The formulation according to the invention, preferably an infusion solution, is aqueous. Since it is intended for parenteral administration, it preferably contains water, saline, dextrose solution or mixture thereof. Preferably, water for injection is the liquid constituent of the formulation according to the invention.

The present invention relates to a sterile ready to use aqueous pharmaceutical formulation comprising flucytosine or its pharmaceutically acceptable salts thereof, wherein the pharmaceutical formulation is free of buffering agent.

In one embodiment, the formulation of the present invention further comprises a pharmaceutically acceptable excipient selected from a tonicity adjusting agent, and a pH adjusting agent.

In another embodiment, the formulation of the present invention comprises about 5 mg/ml to about 20 mg/ml flucytosine or its pharmaceutically acceptable salts thereof.

In a preferred embodiment, the formulation of the present invention comprises about 10 mg/ml flucytosine or its pharmaceutically acceptable salts thereof.

In one other embodiment, the formulation of the present invention comprises sodium chloride as tonicity adjusting agent.

In another embodiment, the formulation of the present invention further comprises a pH adjusting agent. In an embodiment, the formulation of the present invention has a pH between about 6.0 to about 8.0. In a preferred embodiment, the formulation of the present invention has a pH of about 7.5.

In a further embodiment, the formulation of the present invention is manufactured by aseptic fill finish method.

In an embodiment, the formulation of the present invention, when stored for 24 months at 25° C./60% RH contains no more than 0.7% of 5-fluorouracil impurity.

In one more embodiment, the formulation of the present invention, when stored for 24 months at 25° C./60% RH contains no more than 0.2% of ethenoflucytosine impurity.

Another embodiment relates to use of the formulation of the present invention in treatment of systemic yeast and fungal infections.

A further embodiment relates to a sterile ready to use aqueous pharmaceutical formulation comprising about 5 mg/ml to about 20 mg/ml flucytosine or its pharmaceutically acceptable salts thereof, about 8.05 mg/ml sodium chloride, and optionally a pH adjusting agent, wherein the pharmaceutical formulation is free of buffering agent. In a preferred embodiment, the formulation of the present invention comprises about 10 mg/ml flucytosine or its pharmaceutically acceptable salts thereof.

Another embodiment relates to a sterile ready to use aqueous pharmaceutical formulation comprising about 5 mg/ml to about 20 mg/ml flucytosine or its pharmaceutically acceptable salts thereof, about 6.5 mg/ml sodium chloride, and optionally a pH adjusting agent, wherein the pharmaceutical formulation is free of buffering agent. In a preferred embodiment, the formulation of the present invention comprises about 10 mg/ml flucytosine or its pharmaceutically acceptable salts thereof.

The formulation according to the present invention, preferably an infusion solution, can be prepared by conventional processes known to the person skilled in the art.

In one embodiment, the formulation according to the present invention can be prepared by a process, comprising
A) dissolving a tonicity agent and optionally a buffering agent in water for injection under stirring;
B) dissolving flucytosine in solution of step (A); and
C) adjusting the pH of the solution, if required and
D) filtering the solution of step (C) through a 0.2 μm membrane sterile filter and,
E) filling into flexible plastic containers under aseptic conditions.

In another embodiment, the formulation according to the present invention can be prepared by a process, comprising
A) dissolving a tonicity agent and optionally a buffering agent in water for injection under stirring;
B) dissolving flucytosine in solution of step (A); and
C) adjusting the pH of the solution, if required and
D) filling into flexible plastic containers under aseptic conditions and,
E) terminally sterilizing the solution of step (D) by autoclaving.

The sterile ready to use aqueous pharmaceutical formulation of the present invention comprising flucytosine or its pharmaceutically acceptable salts in a therapeutically effective amount and pharmaceutically acceptable excipients having a pH range of from about 6.0 to about 8.0, are dispensed or packaged in a pharmaceutically acceptable container customary for injection-ready solutions or parenteral preparations. The containers made of glass or plastic are preferred such as glass vials, plastic intravenous bags, including pre-mix bags and admix bags. Preferably, the containers are flexible plastic bags, such as are customary for injection-ready solutions. In an embodiment, plastic container is made of polyolefins and are optionally surrounded by a second bag, The formulation of present invention is filled and stored in polymeric containers which are preferably flexible and are made up of polyethylene, polyvinyl chloride or polypropylene. In some embodiments, the formulation of present invention can be aseptically filled and stored in commercially available intravenous bags such as, but are not limited to: ADDEASE®, ADD-VANTAGE®, BFS™, DUPLEX™, EXCEL®, FIRST CHOICE™ GALAXY®, INTRAVIA®, PROPYFLEX™, SOLOMIX®, STEDIM® 71, STEDIM®100, VIAFLEX®, VIAFLO™, and VISIV® and the like. Polymeric containers can further be provided with a moisture barrier as a secondary packaging system to prevent the loss of water during storage and to further ensure the stability of the formulation. A preferred moisture barrier is an aluminium overpouch.

Procedure for filling formulations of the present invention in containers and their subsequent processing is done with care to destroy or eliminate any microorganisms that may be present in the formulations. According to an embodiment, sterile ready to use aqueous pharmaceutical formulation according to the present invention may be prepared using aseptic processing techniques. All the ingredients used for preparing the formulation of present invention are sterile. Sterility is maintained by using sterile materials and a controlled working environment. All containers and apparatus are sterilized, preferably by heat sterilization, prior to filling. The plastic container is then filled under aseptic conditions. Utmost care is taken to avoid the microbiological contamination.

Aseptic sterilization is preferably carried out by passing the formulation through at least one sterile filter, for e.g., 0.22 μm. Several sterilizing grade membrane filters are available for aseptic filtration of water based dosage forms such as cellulose acetate, nylon, polyether sulfone (PES), polypropylene (PP), polyvinyl difluoride (PVDF) and the like.

In a preferred embodiment, the formulation according to the invention is an infusion solution which is prepared for intravenous infusion over a period of time of 2 minutes to 24 hours, more preferably over a period of time of 3 minutes to 6 hours, even more preferably 5 minutes to 1 hour, most preferably 10 minutes to 45 minutes and in particular 20 minutes to 40 minutes.

A further aspect of the invention relates to the formulation comprising flucytosine as described above, preferably an infusion solution, for the treatment of systemic yeast and fungal infections due to sensitive organisms: such infections include cryptococcosis, candidiasis, chromomycosis and infections due to Torulopsis glabrata and Hansenula or the use of flucytosine for the production of a formulation described above for the treatment of systemic yeast and fungal infections due to sensitive organisms: such infections include cryptococcosis, candidiasis, chromomycosis and infections due to Torulopsis glabrata and Hansenula. Preferably the infection is cryptococcal meningitis and severe systemic candidiasis.

As per an embodiment, the formulation of present invention is administered in combination with amphotericin-B or fluconazole as determined by doctor considering the patient's condition.

The dose of pharmaceutical formulation of present invention to be administered to the patient in need thereof depends on the age, sex, body weight and condition of patient and can be determined by doctor. In an embodiment, the formulation of present invention may be directly into a vein, through a central venous catheter, or by intraperitoneal infusion. The daily dosage in adults may be 200 mg/kg bodyweight divided into 3-10 doses over the 24 hours, preferably 4-7 doses over the 24 hours. In patients harboring extremely sensitive organisms, a total daily dose can be adjusted by doctor preferably between 100 to 150 mg/kg bodyweight.

The formulation of present invention is ‘ready-to-use’ parenteral dosage form which does not require dilution before administration, the volume of the aqueous solution may be from about 50 ml to 1000 ml, preferably 100 ml to 300 ml, most preferably 250 mL.

The formulation of present invention exhibits acceptable stability retains a pharmaceutically desirable appearance without any particulate matter, precipitates and crystallization of 5FC. Further, the formulation provided herein, is suitable for parenteral administration.

The following examples are for the purpose of illustration of the invention only and are not intended to limit the scope of the present invention in any manner whatsoever.

Parenteral formulations of flucytosine of the present invention were prepared by using the following methodology:

### Example 1

TABLE 1

| Sr. | | |
|---|---|---|
| No. | Ingredients | Qty (mg/mL) |
| 1 | Flucytosine | 10 |
| 2 | Sodium chloride | 8.05 |
| 3 | water for | Qs to 1 mL |
| | injections | |

### Manufacturing Process:

Dissolve sodium chloride in water for injection (WFI) under stirring to get clear solution. Add flucytosine gradually to solution under continuous stirring. Stir the mixture for 2 hours to ensure uniform mixing of flucytosine in aqueous solution. Adjust the total volume using WFI and stir to ensure uniform mixing. Filter the solution through a 0.22 μm filter to yield a sterile solution and fill the solution under aseptic condition in a suitable container closure system such as glass vials or bags.

### Example 2

| Sr. | | |
|---|---|---|
| No. | Ingredients | Qty (mg/mL) |
| 1 | Flucytosine | 10 |
| 2 | Sodium chloride | 6.50 |
| 3 | water for | Qs to 1 mL |
| | injections | |

### Manufacturing Process:

Dissolve sodium chloride in water for injection (WFI) under stirring to get clear solution. Add flucytosine gradually to solution under continuous stirring. Stir the mixture for 2 hours to ensure uniform mixing of flucytosine in aqueous solution. Adjust the total volume using WFI and stir to ensure uniform mixing. Filter the solution through a 0.22 μm filter to yield a sterile solution and fill the solution under aseptic condition in a suitable container closure system such as glass vials or bags.

The formulation of the present invention was subjected to analytical evaluation like assay and related substances at storage conditions as laid down by the ICH guidelines and below are the results.

| | | | | | | Any | |
|---|---|---|---|---|---|---|---|
| | Time | | Assay | 5-Fluorouracil | Ethenoflucytosine | unspecified | Total |
| | point | pH | (%) | impurity | impurity | impurity | impurity |
| Conditions | Initial | 6.40 | 100.7 | <0.1 | NP | ND | <0.1 |
| 25° C./60% | 3 M | 7.01 | 100.0 | 0.095 | NP | <0.05 | 0.09 |
| RH | 6 M | 7.27 | 102.6 | 0.143 | 0.01 | <0.05 | 0.14 |
| | 9 M | 7.36 | 101.0 | 0.19 | 0.02 | <0.05 | 0.19 |
| | 12 M | 7.34 | 100.9 | 0.23 | 0.03 | ND | 0.26 |
| | 18 M | 7.23 | 100.9 | 0.34 | 0.07 | ND | 0.41 |
| | 24 M | 7.40 | 99.4 | 0.47 | 0.11 | ND | 0.58 |
| 30° C./75% | 3 M | 7.04 | 100.4 | 0.15 | NP | <0.05 | 0.15 |
| RH | 6 M | 7.46 | 101.4 | 0.27 | 0.035 | 0.061 | 0.33 |
| 40° C./75% | 3 M | 7.57 | 99.5 | 0.64 | NP | 0.205 | 0.84 |
| RH | 6 M | 7.73 | 100.9 | 1.28 | 0.032 | 0.547 | 1.83 |

The results from the study indicate that the formulation of the present invention was stable at all temperature conditions and there is no change in pH and assay of solution during the shelf life. It was also seen that the 5-fluorouracil (5-FU) and the ethenoflucytosine impurity was maintained to low levels even at the end of 24 months when stored at 25° C./60% RH. The results demonstrate that the formulations of the present invention remain stable at all conditions, even without the addition of buffer.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to a “cosolvent” refers to a single cosolvent or to combinations of two or more cosolvents, and the like.

1. A sterile ready to use aqueous pharmaceutical formulation comprising flucytosine or its pharmaceutically acceptable salts thereof, wherein the pharmaceutical formulation is free of buffering agent.

2. The formulation of claim 1, wherein the formulation further comprises a pharmaceutically acceptable excipient selected from a tonicity adjusting agent, and optionally a pH adjusting agent.

3. The formulation of claim 1, wherein the formulation comprises about 5 mg/ml to about 20 mg/ml flucytosine or its pharmaceutically acceptable salts thereof.

4. The formulation of claim 1, wherein the formulation comprises about 10 mg/ml flucytosine or its pharmaceutically acceptable salts thereof.

5. The formulation of claim 2, wherein the formulation comprises sodium chloride as tonicity adjusting agent.

6. The formulation of claim 2, wherein the formulation further comprises a pH adjusting agent.

7. The formulation of claim 1, wherein the formulation has a pH between about 6.0 to about 8.0.

8. The formulation of claim 7, wherein the formulation has a pH of about 7.5.

9. The formulation of claim 1, wherein the pharmaceutical formulation is manufactured by aseptic fill finish method.

10. The formulation of claim 1, wherein the formulation, when stored for 24 months at 25° C./60% RH contains no more than 0.7% of 5-fluorouracil impurity.

11. The formulation of claim 1, wherein the formulation, when stored for 24 months at 25° C./60% RH contains no more than 0.2% of ethenoflucytosine impurity.

12. The formulation of claim 1, for use in treatment of systemic yeast and fungal infections.

13. A sterile ready to use aqueous pharmaceutical formulation comprising about 5 mg/ml to about 20 mg/ml flucytosine or its pharmaceutically acceptable salts thereof, about 8.05 mg/ml sodium chloride, and optionally a pH adjusting agent, wherein the pharmaceutical formulation is free of buffering agent.

14. The formulation of claim 13, wherein the formulation comprises about 10 mg/ml flucytosine or its pharmaceutically acceptable salts thereof.

15. A sterile ready to use aqueous pharmaceutical formulation comprising about 5 mg/ml to about 20 mg/ml flucytosine or its pharmaceutically acceptable salts thereof, about 6.5 mg/ml sodium chloride, and optionally a pH adjusting agent, wherein the pharmaceutical formulation is free of buffering agent.

16. The formulation of claim 15, wherein the formulation comprises about 10 mg/ml flucytosine or its pharmaceutically acceptable salts thereof.

17. The formulation of claim 1, which is free of buffering agent selected from the group consisting of sodium citrate, potassium citrate, acetate, phosphate, malate, lactate, glutamate, titrate, benzoate, glycine, tromethamine (Tris), and mixtures thereof.

18. The sterile ready to use aqueous pharmaceutical formulation of claim 13, which is free of buffering agent selected from the group consisting of sodium citrate, potassium citrate, acetate, phosphate, malate, lactate, glutamate, titrate, benzoate, glycine, tromethamine (Tris), and mixtures thereof.

19. The sterile ready to use aqueous pharmaceutical formulation of claim 15, which is free of buffering agent selected from the group consisting of sodium citrate, potassium citrate, acetate, phosphate, malate, lactate, glutamate, titrate, benzoate, glycine, tromethamine (Tris), and mixtures thereof.

20. The formulation of claim 1, that is aseptic filled and stored in a pre-sterilized storage container suitable for parenteral administration.

21. The sterile ready to use aqueous pharmaceutical formulation of claim 13, that is aseptic filled and stored in a pre-sterilized storage container suitable for parenteral administration.

22. The sterile ready to use aqueous pharmaceutical formulation of claim 15, that is aseptic filled and stored in a pre-sterilized storage container suitable for parenteral administration.

23. The formulation of claim 1, that is aseptic filled and stored in a pre-sterilized flexible plastic container suitable for parenteral administration.

24. The sterile ready to use aqueous pharmaceutical formulation of claim 13, that is aseptic filled and stored in a pre-sterilized flexible plastic container or glass vial, suitable for parenteral administration.

25. The sterile ready to use aqueous pharmaceutical formulation of claim 15, that is aseptic filled and stored in a pre-sterilized flexible plastic container or glass vial, suitable for parenteral administration.
